(19) United States
(12) Patent Application Publication
(10) Pub. No.: US 2022/0296478 A1
(43) Pub. Date: Sep. 22, 2022

(54) Single Use Topical Solution Assembly

(71) Applicant: Braden Stanford, Coronado, CA (US)

(72) Inventor: Braden Stanford, Coronado, CA (US)

(21) Appl. No.: 17/203,093

(22) Filed: Mar. 16, 2021

(51) Int. Cl. A61K 8/11; A61Q 17/00

(52) U.S. Cl. CPC A61K 8/11; A61Q 17/005; A61K 2800/70; A61K 2800/87

(57) ABSTRACT

A single use topical solution assembly for single use hand sanitizing includes a capsule that is comprised of rupturable material. In this way the capsule can be ruptured by squeezing between a user's hands. A topical solution is contained within the capsule. The topical solution is released from the capsule when the capsule is ruptured. In this way the topical solution can sanitize the user's hands when the capsule is squeezed between the user's hands.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to topical solution devices and more particularly pertains to a new topical solution device for single use topical solution application.

### (2) Description of Related Art Including Information Disclosed Under 37 CFR 1.97 and 1.98

The prior art relates to topical solution devices including a variety of capsule-like containers that contain a hand sanitizing solution and which can be ruptured by squeezing between hands. In each case the fluid volume of the capsule-like container, if disclosed, is less than 3.0 ml.

### BRIEF SUMMARY OF THE INVENTION

An embodiment of the disclosure meets the needs presented above by generally comprising a capsule that is comprised of rupturable material. In this way the capsule can be ruptured by squeezing between a user's hands. A topical solution is contained within the capsule. The topical solution is released from the capsule when the capsule is ruptured. In this way the topical solution can sanitize the user's hands when the capsule is squeezed between the user's hands.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a single use topical solution assembly according to an embodiment of the disclosure.

FIG. 2 is a cross sectional view taken along line 2-2 of FIG. 1 of an embodiment of the disclosure.

FIG. 3 is a perspective in-use view of an embodiment of the disclosure.

### DETAILED DESCRIPTION OF THE INVENTION

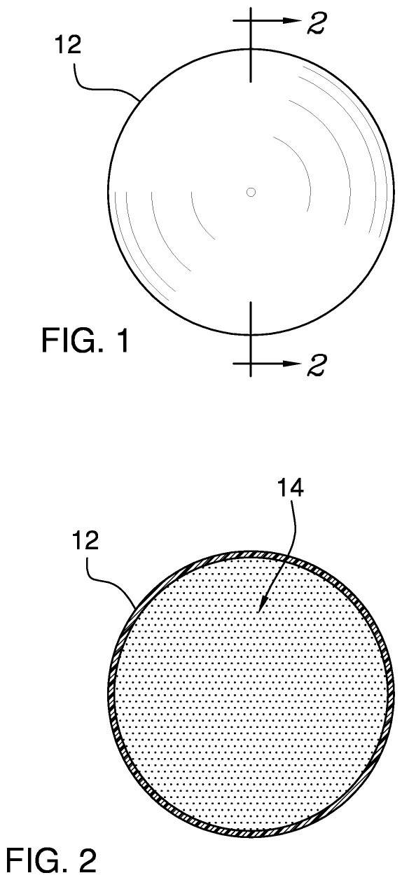

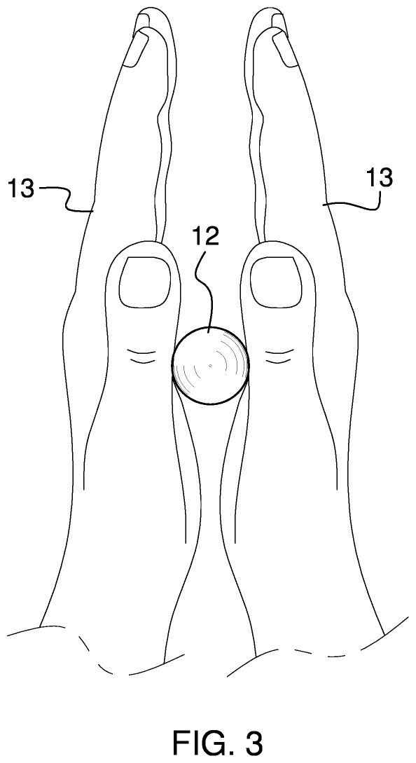

With reference now to the drawings, and in particular to FIGS. 1 through 3 thereof, a new topical solution device embodying the principles and concepts of an embodiment of the disclosure and generally designated by the reference numeral 10 will be described.

As best illustrated in FIGS. 1 through 3, the single use topical solution assembly 10 generally comprises a capsule 12 is that is comprised of rupturable material thereby facilitating the capsule 12 to be ruptured by squeezing between a user's hands 13. Additionally, the capsule 12 has an internal volume of at least 3.0 ml. The capsule 12 is comprised of semi-synthetic vegetable material such that the capsule 12 is safe for use on the human body. The semi-synthetic vegetable material may comprise hypromellose or other similar vegetable based material that is commonly employed for producing dissolvable capsules which are approved for human ingestion, such as oral medication. In this way supply chain problems that can arise from limited availability of plastic bottles, such as those experienced with the existing production manner of topical solutions, can be avoided.

A topical solution 14 is provided and the topical solution 14 is contained within the capsule 12. The topical solution 14 is released from the capsule 12 when the capsule 12 is ruptured. In this way the topical solution 14 can sanitize the user's hands 13 when the capsule 12 is squeezed between the user's hands 13. The capsule 12 contains at least 3.0 ml of the topical solution 14. Moreover, the topical solution may comprise a hand sanitizer such that the fluid volume of the topical solution 14 that is released conforms to the recommendations of the Center for Disease Control (CDC) with respect to hand sanitization to control the spread of Covid-19 and other infectious diseases. Thus, the topical solution 14 can adequately sanitize the user's hands 13 of bacteria for the purposes of inhibiting the transmission of infectious disease. The topical solution 14 may be a solution of ethyl alcohol, isopropyl alcohol, benzalkonium chloride or any other solution approved for the use of hand sanitization that does not contain methanol or 1-propanol. Additionally, the topical solution 14 may comprise any fluid solution that is approved to be used on human skin.

In use, a single capsule 12 is squeezed between the user's hands 13 when the user wishes to sanitize their hands 13. In this way at least 3.0 ml of the topical solution 14 is released onto the user's hands 13. Thus, the user's hands 13 are sufficiently sanitized in accordance with CDC guidelines. Moreover, producing the topical solution 14 in the capsule 12s avoids potential supply chain problems that can arise with the use of plastic bottles. In this way the supply of available personal use, topical solution 14 can be maintained at sufficient production quantities during a pandemic.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A single use topical solution assembly for containing and subsequently releasing a single use volume of topical solution, said assembly comprising:
a capsule being comprised of rupturable material wherein said capsule is configured to be ruptured by squeezing between a user's hands; and
a topical solution being contained within said capsule, said topical solution being released from said capsule when said capsule is ruptured wherein said topical solution is configured to sanitize the user's hands when said capsule is squeezed between the user's hands.

2. The assembly according to claim 1, wherein said capsule is comprised of semi-synthetic vegetable material wherein said capsule is configured to be safe for use on the human body.

3. (canceled)

4. The assembly according to claim 2, wherein said capsule has an internal volume of at least 3.0 ml.

5. The assembly according to claim 4, wherein said capsule contains at least 3.0 ml of said topical solution wherein said topical solution is configured to adequately sanitize the user's hands of bacteria for the purposes of inhibiting the transmission of infectious disease.

6. A single use topical solution assembly for containing and subsequently releasing a single use volume of topical solution, said assembly comprising:
a capsule being comprised of rupturable material wherein said capsule is configured to be ruptured by squeezing between a user's hands, said capsule having an internal volume of at least 3.0 ml, said capsule being comprised of semi-synthetic vegetable material wherein said capsule is configured to be safe for use on the human body; and
an topical solution being contained within said capsule, said topical solution being released from said capsule when said capsule is ruptured wherein said topical solution is configured to sanitize the user's hands when said capsule is squeezed between the user's hands, said capsule containing at least 3.0 ml of said topical solution wherein said topical solution is configured to adequately sanitize the user's hands of bacteria for the purposes of inhibiting the transmission of infectious disease.

7. A method of dispensing a single use volume of topical solution on a user's hands, the steps of the method comprising:
providing a capsule being comprised of semi-synthetic vegetable material and having an internal volume of at least 3.0 ml;
providing a topical solution;
filling said capsule with said topical solution; and
squeezing said capsule between a user's hands thereby rupturing said capsule to release said topical solution on the user's hands wherein said topical solution is configured to adequately sanitize the user's hands for the purposes of inhibiting the transmission of infectious diseases.
